(19) United States
(12) Patent Application Publication
(10) Pub. No.: US 2025/0268807 A1
(43) Pub. Date: Aug. 28, 2025

(54) ANTI-AGING PEPTIDE CONTAINING COSMETIC

(71) Applicant: SBLA BRANDS INC., Boca Raton, FL (US)

(72) Inventor: Randi SHINDER, Boca Raton, FL (US)

(73) Assignee: SBLA BRANDS INC., Boca Raton, FL (US)

(21) Appl. No.: 18/859,211

(22) PCT Filed: Apr. 26, 2023
(86) PCT No.: PCT/US2023/020072

§ 371 Date: Oct. 23, 2024

Related U.S. Application Data
(60) Provisional application No. 63/335,384, filed on Apr. 27, 2022.

(51) Int. Cl. A61K 8/64; A61K 8/49; A61K 8/67; A61K 8/73; A61Q 19/08

(52) U.S. Cl. CPC A61K 8/64; A61K 8/4953; A61K 8/675; A61K 8/676; A61K 8/735; A61Q 19/08

(57) ABSTRACT

The invention provides an anti-aging peptide containing cosmetic. Also provided are methods of reducing the appearance of aging on the skin, especially skin around the eye.

### FIELD OF THE INVENTION

This invention pertains generally to anti-aging cosmetics and, more particularly to anti-aging peptide containing cosmetic especially formulated for application to skin around the eye.

### BACKGROUND OF THE INVENTION

As the skin ages, there are physiological and physical changes to the skin that result in an appearance that is characteristic of the aging skin. Compared to youthful skin, aging skin appears to be dry, rough, dull, lusterless, with increased wrinkles and decreased elasticity.

The physical changes to skin that result from the aging process can have a negative effect on the individual in part due to societal pressures to maintain a youthful appearance.

There exists a need for improved over-the-counter products effective at mitigating the effect of time on skin.

### SUMMARY OF THE INVENTION

An object of the present invention is to provide an anti-aging peptide containing cosmetic. In accordance with an aspect of the present invention, there is provided an anti-aging cosmetic comprising an arginine/lysine polypeptide. In some embodiments, the anti-aging cosmetic further comprises one or more of oligopeptide-10, niacinamide, vitamin C, caffeine, hyaluronic acid, and acetyl glucosamine.

In some embodiments, the anti-aging cosmetic is formulated for application to skin around the eye and comprises arginine/lysine peptide, oligopeptide-10, niacinamide, vitamin C, caffeine, hyaluronic acid, and acetyl glucosamine.

In accordance with another aspect of the invention, there is provided a method of reducing the effect of aging on skin, the method comprising applying the anti-aging cosmetic of the invention at least once a day to the skin, preferably twice a day.

In some embodiments, the invention provides a method of reducing the signs of aging on skin around the eye and opens the eye area by lifting eyelids, visibly smooths eyelid creases, tightens the skin of the entire eye area, builds collagen and regenerates skin cells, brightens skin, reduces under eye puffiness and/or improves skin texture and discoloration.

### BRIEF DESCRIPTION OF THE DRAWINGS

These and other features of the invention will become more apparent in the following detailed description in which reference is made to the appended drawings.

FIG. 1 is a series of before (top) and after (bottom) pictures of treated skin of the eye of multiple test subjects. Images are not retouched.

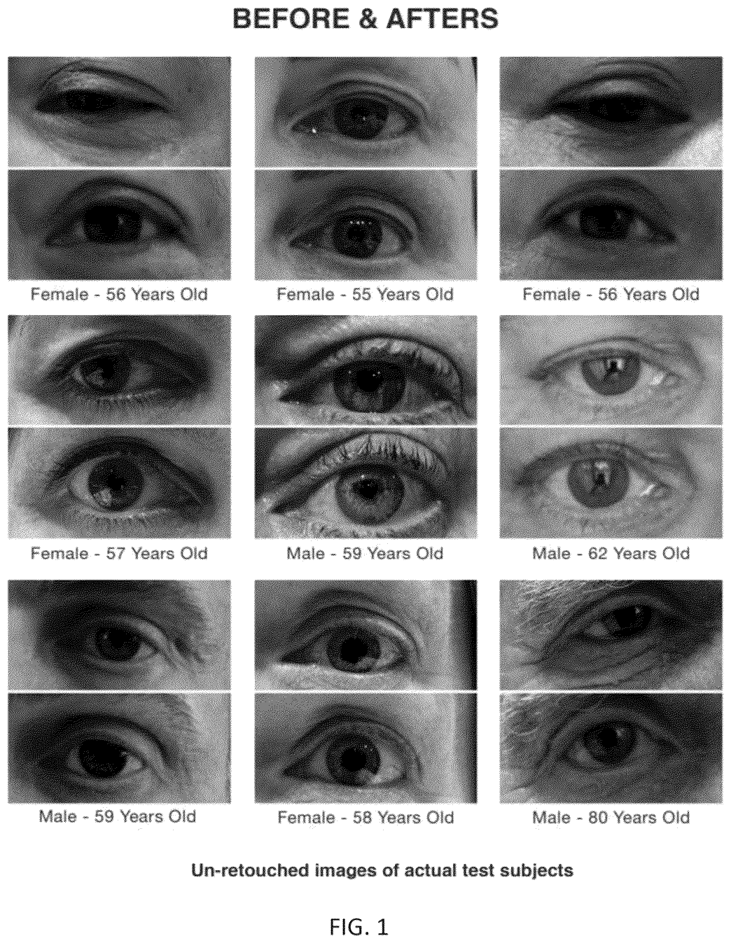

### DETAILED DESCRIPTION OF THE INVENTION

The invention relates to an anti-aging cosmetic that includes peptides. These cosmetics may be in various forms such as creams, aqueous emulsions, gels, aqueous solutions, etc. In some embodiments, the cosmetics are specifically formulated for application to the skin around the eye area and are optionally formulated for application using a specific applicator, for example, a rolling ball applicator.

In some embodiments, the peptides of the invention act to improve the appearance of aging skin by promoting collagen and/or elastin production. The peptides of the invention comprise arginine and/or lysine, preferably L-arginine and/or L-lysine. In some embodiments, the peptides of the invention include a plurality of different peptides comprising arginine and/or lysine. The peptides of the invention are generally between 2 and 50 amino acids in length. In some embodiments, the peptides are shorter than 25 amino acids. In alternative embodiments, the peptides are 10 amino acids in length. In some embodiments, the cosmetic includes a peptide mixture comprising oligomers of L-arginine and/or L-lysine.

Optionally, the peptides of the invention include other amino acids present in high amounts in collagen including proline and glycine and/or other amino acids shown to promote collagen production.

Optionally, free L-arginine and/or L-lysine and/or L-proline and/or hydroxyproline and hydroxylysine and/or collagen peptides are included in the cosmetic.

In some embodiments, the anti-aging cosmetic comprises oligopeptide-10. Oligopeptide-10 has the amino acid sequence: Phe-Ala-Lys-Ala-Leu-Lys-Ala-Leu-Leu-Lys-Ala-Leu-Lys-Ala-Leu.

Oligopeptide-10 has anti-microbial properties and significantly reduces the appearance of blemishes and red, blotchy spots.

In some embodiments, the anti-aging cosmetic further comprises a humectant or moisturizing agent. Appropriate humectants or moisturizing agents are known in the art and include hyaluronic acid. The hyaluronic acid is provided in amount sufficient to moisturize the skin and optionally is about 0.01% to about 10% by weight, preferably about 0.5% to about 3% by weight, and still more preferrable about 0.5% to about 2% by weight based on the total weight of the cosmetic.

In some embodiments, the anti-aging cosmetic further comprises vitamin C optionally with vitamin E and/or ferulic acid. The vitamin C may be provided as L-ascorbic acid or ascorbic acid. In some embodiments, the vitamin C is formulated to improve stability, such formulations are known in the art. Optionally, the vitamin C is about 1% to about 10% by weight, preferable about 5% to about 10% by weight and more preferably about 8% to about 10% by weight based on the total weight of the cosmetic.

In some embodiments, the anti-aging cosmetic, further comprises caffeine. Caffeine is known to promote lipolysis when applied to the skin and may reduce the appearance of puffiness. The caffeine is about 1% to about 10% by weight, preferably about 1% to about 3% by weight based on the total weight of the cosmetic. In some embodiments, niacinamide is provided to in the cosmetic to prevent precipitation of the caffeine at low temperatures.

The niacinamide is about 0.5% to about 10% by weight based on total weight of the cosmetic. In some embodiments, the amount of niacinamide provided is optimized to reduce caffeine precipitation.

Niacinamide also has a whitening or brightening effect on the skin and therefore may be provided in cosmetics without caffeine.

In some embodiments, the anti-aging cosmetic, further comprises acetyl glucosamine. The acetyl glucosamine is about 0.01% to about 25.0% by weight; preferably about 0.5% to about 10.0% by weight, and more preferably about 1.0% to 5.0% by weight based on total weight of the cosmetic.

In further embodiments, the anti-aging cosmetic further comprises tints, pigments and/or sunscreens.

The invention further provides method of reducing the effect of aging on skin. The method comprising applying the anti-aging cosmetic of the invention to the skin at least once a day preferably twice a day.

In some embodiments, the anti-aging cosmetic is used to improve the appearance of the skin around the eye and when applied over a period of time opens the eye area by lifting eyelids visibly smooths eyelid creases, tightens the skin of the entire eye area, builds collagen and regenerates skin cells, brightens skin, reduces under eye puffiness and/or improves skin texture and discoloration.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. An anti-aging cosmetic comprising an arginine/lysine peptide.

2. The anti-aging cosmetic of claim 1, further comprising Oligopeptide 10.

3. The anti-aging cosmetic of claim 2, further comprising a humectant or moisturizing agent.

4. The anti-aging cosmetic of claim 3, wherein the humectant or moisturizing agent is hyaluronic acid.

5. The anti-aging cosmetic of claim 4, wherein the hyaluronic acid is about 0.01% to about 10% by weight, preferably about 0.5% to about 3% by weight, and still more preferrable about 0.5% to about 2% by weight based on the total weight of the cosmetic.

6. The anti-aging cosmetic of claim 5, further comprising vitamin C.

7. The anti-aging cosmetic of claim 6, wherein the vitamin C is provided as L-ascorbic acid or ascorbic acid.

8. The anti-aging cosmetic of claim 7, wherein the vitamin C is about 1% to about 10% by weight, preferable about 5% to about 10% by weight and more preferably about 8% to about 10% by weight based on the total weight of the cosmetic.

9. The anti-aging cosmetic of claim 8, further comprising caffeine.

10. The anti-aging cosmetic of claim 9, wherein the caffeine is about 1% to about 10% by weight, preferably about 1% to about 3% by weight based on the total weight of the cosmetic.

11. The anti-aging cosmetic of claim 9, further comprising niacinamide.

12. The anti-aging cosmetic of claim 11, wherein the niacinamide is about 0.5% to about 10% by weight based on total weight of the cosmetic.

13. The anti-aging cosmetic of claim 11, further comprising acetyl glucosamine.

14. The anti-aging cosmetic of claim 13, wherein the acetyl glucosame is about 0.01% to about 25.0% by weight; preferably about 0.5% to about 10.0% by weight, and more preferably about 1.0% to 5.0% by weight based on total weight of the cosmetic.

15. The anti-aging cosmetic of claim 13, comprising the arginine/lysine peptide, the oligopeptide-10, the niacinamide, the vitamin C, the caffeine, the hyaluronic acid, and the acetyl glucosamine.

16. The anti-aging cosmetic of claim 15, formulated for application to skin around the eye.

17. A method of reducing the effect of aging on skin, the method comprising applying the anti-aging cosmetic of claim 15 at least once a day to the skin.

18. The method of claim 17, wherein the skin is skin around the eye.

19. The method of claim 18, wherein the method opens the eye area by lifting eyelids visibly smooths eyelid creases, tightens the skin of the entire eye area, builds collagen and regenerates skin cells, brightens skin, reduces under eye puffiness and/or improves skin texture and discoloration.
